(19) United States
(12) Patent Application Publication
(10) Pub. No.: US 2003/0093018 A1
(43) Pub. Date: May 15, 2003

(54) Orthesis comprising a flexion and an extension stop that can be adjusted by means of rail pivoting movements

(76) Inventors: Erich Albrecht, Neubeuem (DE); Hans-Georg Opahle, Rosenhelm (DE)

Correspondence Address: Nixon & Vanderhye, 8th Floor, 1100 North Glebe Road, Arlington, VA, 22201-4714, US

(21) Appl. No.: 10/203,557

(22) PCT Filed: Feb. 8, 2001
(86) PCT No.: PCT/EP01/01364

(30) Foreign Application Priority Data
Feb. 10, 2000 (DE) 100 05 764.0

(51) Int. Cl. A61F 5/00

(52) U.S. Cl. 602/16

(57) ABSTRACT

The aim of the invention is to eliminate deficits of movement in the joint area. An orthesis comprising a spring force mechanism that acts in the flexion direction and in the extension direction is provided. The limits of the pivoting area can be adjusted by means of rail pivoting movements. The flexion stop (26) is arranged on a flexion stop carrier disc (28) and the extension stop (27) is arranged on an extension stop carrier disc (29). The movement of rotation of the flexion and extension stop carrier discs (28, 29) can be blocked by means of clamping rings (30, 31). In the unblocked state, the flexion stop (26) and the extension stop (27) can be moved up to the desired limits of the pivoting area by means of a stop carrier device.

The invention relates to an orthesis for reduction of extension and/or bending deficits in the area of a joint, especially an elbow rail, as claimed in the preamble of claim 1.

In particular, joint capsules and/or connective tissue for example after ligament surgery, accidents, inflammations, etc. often have an extension and/or bending deficit. This means that a distal extremity, for example the lower arm, can no longer be moved completely into its normal extension or flexion position with respect to a proximal extremity, for example the upper arm.

In order to counteract this extension or bending deficit, attempts have been made to stretch the contractions or shrinkages again by moving the distal extremity with respect to the proximal extremity by means of a Quengel device under spring tension in the form of an orthesis in the flexion direction as far as a certain flexion stop and in the extension direction as far as a certain extension stop. With increasing mobilization of the joint or the corresponding tendons and ligaments the flexion and extension stop is adjusted accordingly.

Known ortheses used for Quengel applications for limiting the swivelling area have stop pins which can be inserted into various holes located around the swivelling axis. The disadvantage here is that the flexion and extension stop can only be adjusted in rough increments, for example 15 degree increments, and the limits of the swivelling range therefore often cannot be adjusted accurately enough.

The object of the invention is to devise an orthesis for reduction of extension and/or bending deficits with which the flexion and extension stop can be continuously adjusted in a simple, fast and accurate manner.

This object is achieved as claimed in the invention by the features of claim 1. Advantageous embodiments of the invention are described in the other claims.

In the orthesis as claimed in the invention the flexion stop is located on a flexion stop carrier disk which can be turned around the swivelling axis of the rail hinge and the extension stop is located on the extension stop carrier disk which can be turned around the swivelling axis of the rail hinge. The flexion and extension stop carrier disks are dynamically connected to a releasable locking means in order to block or enable the rotation of the flexion and extension stop carrier disk around the swivelling axis. Furthermore, there is a stop driver means which is coupled torsionally strong to the first rail and which can turn together with it around the swivelling axis, and with which with the locking means released the flexion stop can be moved by swivelling the first rail as far as the desired limit of the flexion swivelling range and the extension stop can be moved by swivelling the first rail as far as the desired limit of the extension swivelling range. By locking the locking means the flexion stop can be locked at the limit of the flexion swivelling range and the extension stop can be locked at the limit of the extension swivelling range.

For the orthesis as claimed in the invention it is thus characteristic that adjustment of the flexion stop at the limit of the flexion swivelling range and of the extension stop at the limit of the extension swivelling range is possible without any tools and without pulling out and inserting any stop pins. Rather it is possible solely by swivelling the first rail as far as the limit of the flexion swivelling range to move the flexion stop up to this limit, and the extension stop by swivelling the first rail as far as the limit of the extension swivelling range, whereupon a locking means is actuated which locks the flexion (and extension stop at the selected limits of the swivelling range. The first rail can thereupon only be swivelled relative to the second rail within the fixed limits of the swivelling range. If the flexion stop and/or extension stop must be readjusted in the course of treatment, it is simply necessary to release the locking means, whereupon by swivelling the first rail as far as the new swivelling range limit the corresponding stop is entrained as far as this swivelling range limit and then can be fixed again by the locking means.

The orthesis as claimed in the invention thus has an integrated stop driver means which is connected torsionally strong to the first rail and when the first rail is swivelled moves the flexion and extension stop into the desired position.

According to one advantageous embodiment of the invention, the stop driver means which is coupled torsionally strong to the first rail is made as a swivelling range limitation means which at the limit of the flexion swivelling range strikes the flexion stop and at the limit of the extension swivelling range strikes the extension stop. In other words, this means that the stop driver means, in addition to the function of moving the flexion and extension stop with the locking means released to the swivelling range limits, has the further function of preventing the first rail from moving over the set swivelling range limits with the flexion and extension stop locked.

According to one advantageous embodiment, the stop driver means consists of at least one stop driver disk with a lengthwise slot which is routed in an arc shape around the swivelling axis and into which the flexion stop and extension stop project.

Feasibly there is a return spring which engages the flexion stop carrier disk and the extension stop carrier disk in order to return the flexion stop and extension stop to their initial location when the locking means has been released.

According to one advantageous embodiment the locking means consists of a clamping means which is held on the second rail, which extends over the flexion stop carrier disk and the extension stop carrier disk, and which can be moved by a clamp actuating means between the clamp position and the release position.

Here the clamp means feasibly consists of two clamping rings which extend over the flexion stop carrier disk and the extension stop carrier disk on their outer peripheral surface with narrow play and in the clamped position prevent rotation of the flexion stop carrier disk and the extension stop carrier disk.

The invention is detailed below by way of example using the drawings.

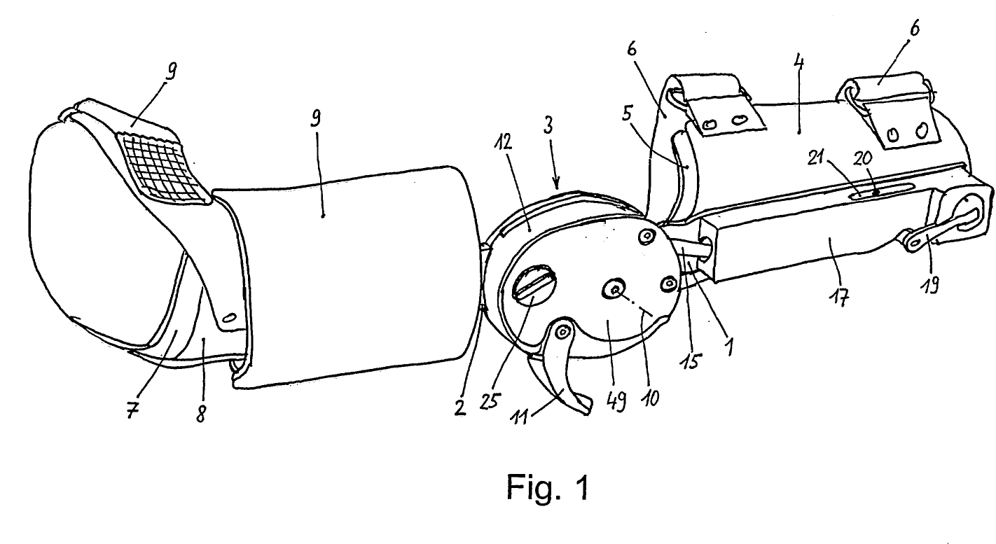

FIG. 1 shows a perspective view of the orthesis as claimed in the invention;

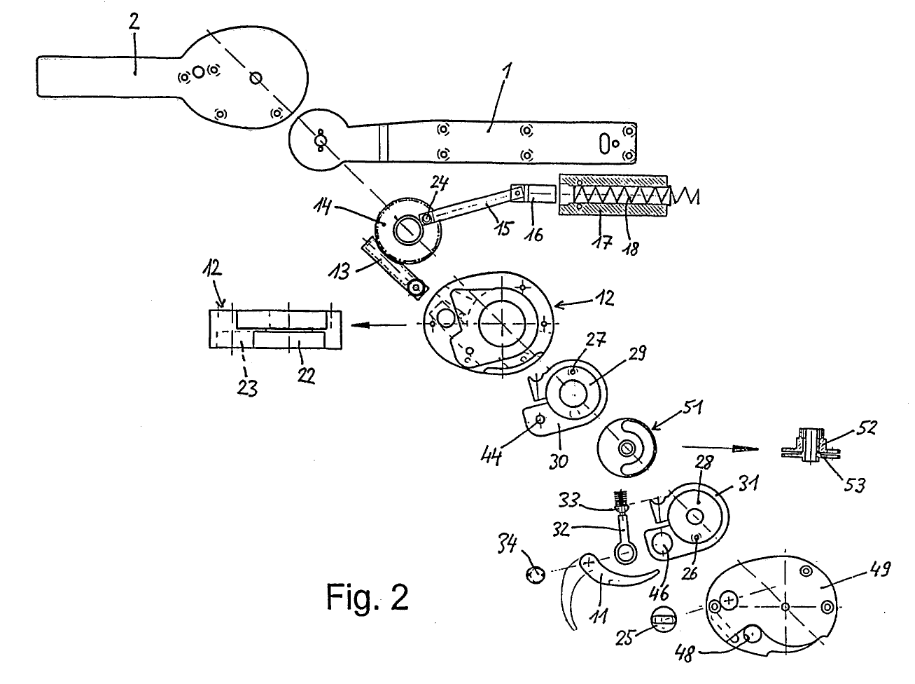

FIG. 2 shows an exploded view of important parts of FIG. 1;

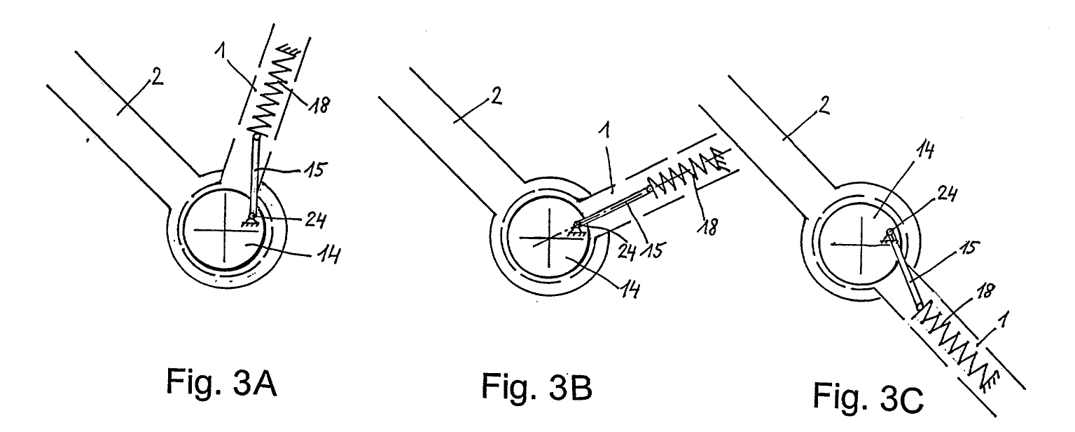

FIGS. 3A-3C show schematics for illustrating how the bilaterally acting spring force mechanism of the orthesis from FIG. 1 works;

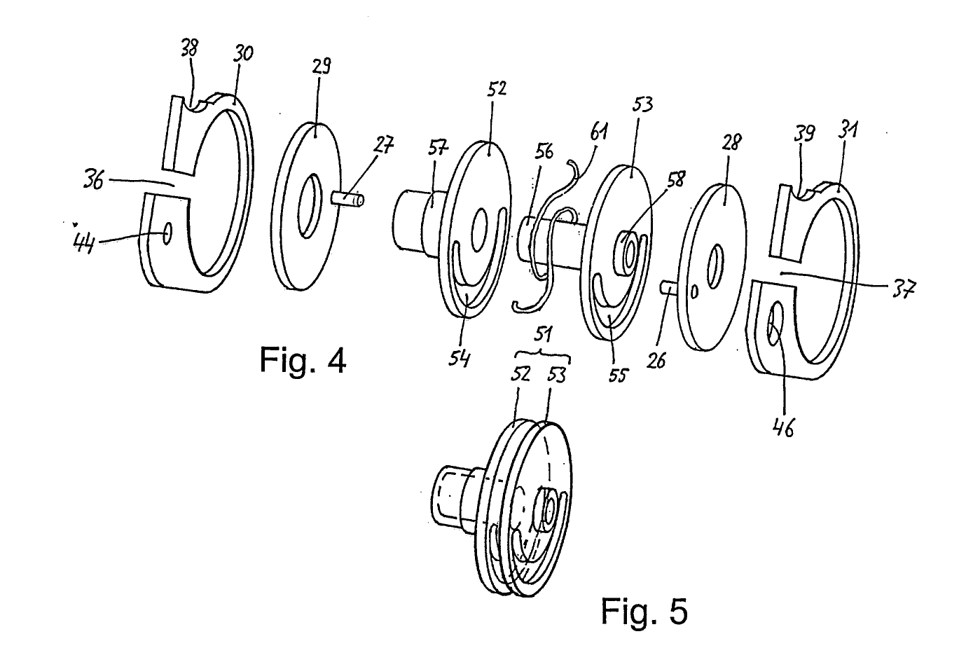

FIG. 4 shows a perspective exploded view of the locking means in the form of a two clamping rings and the flexion and extension stop carrier disks and stop driver means;

FIG. 5 shows the stop driver means from FIG. 4 in the assembled state;

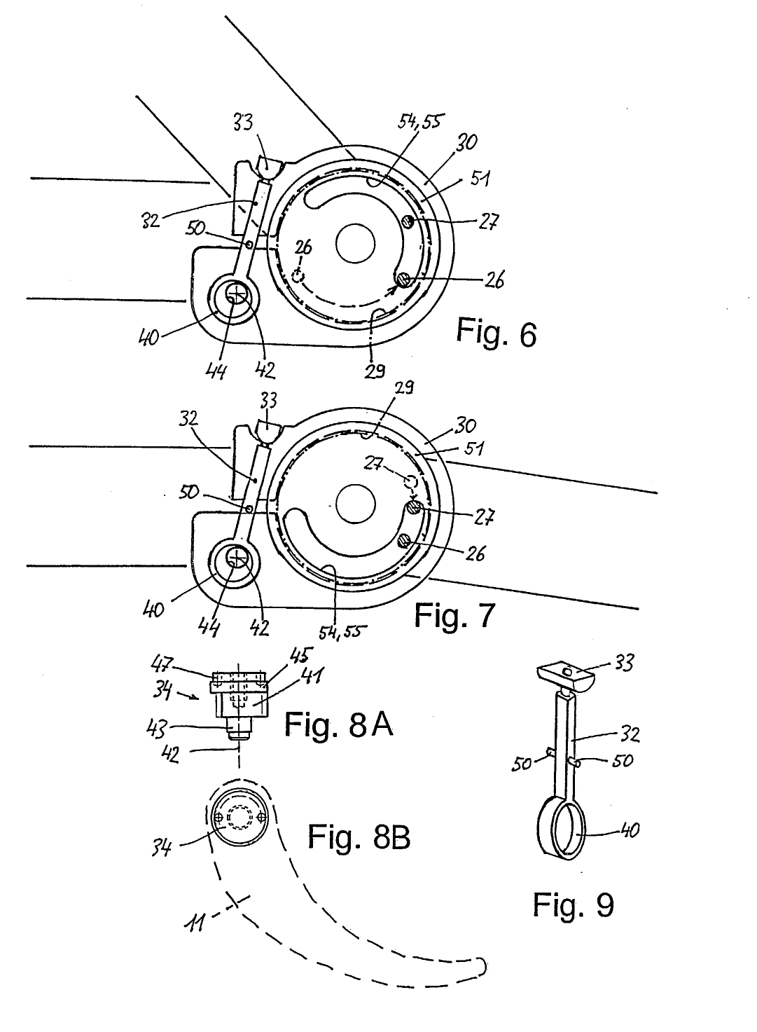

FIG. 6 shows the orthesis in the maximum flexion position, the stop driver means striking the flexion stop;

FIG. 7 shows the orthesis in the maximum extension position, the stop driver means striking the extension stop;

FIG. 8A shows a side view of the eccentric element for actuating the locking means;

FIG. 8B shows an overhead view of the eccentric element from FIG. 8A and the clamp lever connected to it;

FIG. 9 shows a perspective view of the clamping rod; and

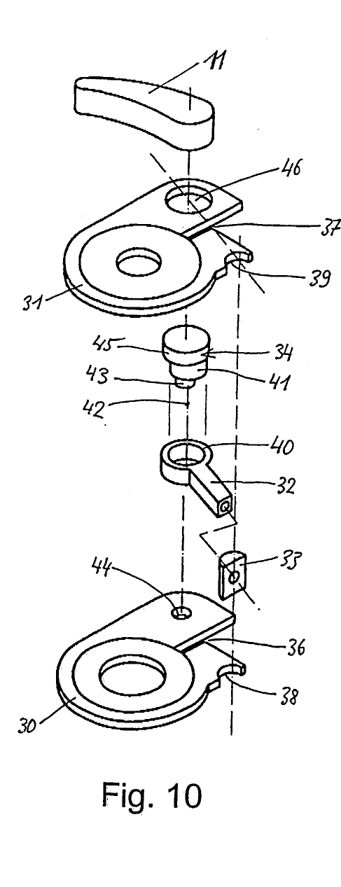

FIG. 10 shows an exploded view of the locking means.

The orthesis as claimed in the invention is detailed below using an elbow orthesis. Other possible applications, for example as a knee joint orthesis, are however easily conceivable.

The orthesis shown in FIG. 1 has a first rail 1 which is attached distally, i.e. to the forearm, and a second rail 2 which is attached proximally, i.e. to the upper arm, and is hinged to a first rail 1 via a rail hinge 3. The orthesis is located radially when used as an elbow rail. A laterally arranged rail pair which is used simply as a “co-traveller” and which does not have a spring force mechanism is not necessary in an application as an elbow rail, but could be provided if this should be desirable for example for reasons of stability. If the orthesis is used as a knee joint orthesis, it is advantageous to likewise provide on the opposite side of the knee joint a rail pair which is connected via the corresponding shells and/or holding belts to the orthesis shown and the extremities.

To attach the first rail 1 to the forearm, a plastic half shell 4 is attached to the first rail 1 and is lined with a cushion material 5, for example a foam material. The forearm is inserted into the half shell 4 which is attached to the forearm with attachment straps 6 with velcro closure.

To attach the second rail 2 to the upper arm the second rail 2 likewise has a plastic half shell 8 which is lined with a cushion material 7 and which is attached to the upper arm with attachment straps 9 with velcro closure.

The first rail 1 is hinged to the second rail 2, the swivelling axis being labelled with reference number 10. Furthermore, the orthesis has a double-acting spring force mechanism which applies a pretensioning force to the orthesis both in the flexion and extension direction. The switching of the pretensioning force from the flexion direction to the extension direction and vice versa takes place automatically here starting with a certain, adjustable swivelling angle, as will be detailed later. Furthermore, the orthesis has a mechanism which makes it possible to continuously adjust the flexion stop which is used to limit the swivelling range in the flexion direction, and an extension stop which is used to limit the swivelling range in the extension direction, in a simple, quick and very exact manner. The adjustment takes place here by the swivelling motion of the first rail 1 relative to the second rail 2 as far as the desired limit of the swivelling range, the flexion and extension stop being entrained as far as the respective limit of the swivelling range and there it can be locked by means of a locking means which is simple to actuate and which is actuated via a clamping lever. This is also detailed below.

As is apparent from FIG. 2, the second rail 2 which is to be placed proximally forms the rear termination of a housing 12 which in FIG. 2 is shown both in a side representation and also next to it to the left in a center horizontal section. Directly next to the second rail 2 is the first rail 1 which is to be placed distally. This first rail 1 in the axial direction adjoins the double-acting spring force mechanism which consists of a worm drive/worm wheel drive with a worm 13 and a worm wheel 14, a connecting rod 15 which is hinged to the worm wheel 14, a spring housing 17 which is attached to the first rail 1 and a compression spring 18 which is guided in the spring housing 17 and which exerts a pretensioning force on the piston 16 and thus on the connecting rod 15. The end of the compression spring 18 which is opposite with respect to the piston 16 can be pushed by means of a crank 19 (FIG. 1), which can be folded out and which is connected to an angular gear which is not shown and an adjusting piston, along the spring housing 17 in order to set the pretensioning of the compression spring 18 to the desired value. The adjusted position of the back end of the compression spring 18 and thus the level of the pretensioning force are indicated by an indicator pin 20 (FIG. 1) which is connected to the rear adjusting piston and which can be viewed through a slot 21 in the spring housing 17.

The worm wheel 14 is pivotally supported in a rear cavity 23 of the housing 12. The worm 13 is likewise supported in a rear cavity 23 which is shown simply by a broken line such that it can be turned around its lengthwise axis. Since the worm wheel 14 engages the worm 13 and it is a self-locking gear, the set angular position of the worm wheel 14 and thus the location of the coupling point 24 of the connecting rod 15 relative to the housing 12 and relative to the second rail 2 which is permanently connected to the housing 12 are maintained as long as the worm 13 is not turned. The location of the coupling point 24 relative to the second rail 2 determines dead center, i.e. the swivelling angle, starting from which the spring force mechanism switches the orthesis from flexion to extension and vice versa. This is detailed later.

The worm drive/worm wheel drive is adjusted by turning a knob 25 (FIG. 1) which is dynamically connected to the worm 13 via gearing which is not detailed. When the knob 25 is turned the worm 13 is turned around its lengthwise axis, by which the worm wheel 14 is likewise turned and the coupling point 24 changes its angular position relative to the second rail 2.

The free swivelling range of the first rail 1 relative to the second rail 2 is limited in the flexion direction by the flexion stop 26 and in the extension direction by the extension stop 27 (FIGS. 2, 4, 6, and 7) in the form of transverse pins. As is especially apparent from FIGS. 2 and 4, the flexion stop 26 is attached off-center to the flexion stop carrier disk 28 and the extension stop 27 to the extension stop carrier disk 29 and they project in the transverse direction, i.e. parallel to the swivelling axis 10. The flexion and extension stop carrier disks 28, 29 can be turned independently of one another around the swivelling axis 10 in the unlocked state so that the angular position of the flexion stop 26 and of the extension stop 27 relative to the housing 12 and thus to the second rail 2 can be changed and adjusted in the desired manner. If the flexion stop 26 and extension stop 27 are located at the desired location, the flexion and extension stop carrier disks 28, 29 are locked relative to the housing 12 and thus to the second rail 2. This locking takes place by a locking means which is detailed in FIGS. 4 and 10 and which consists of clamping rings 30, 31 which surround the flexion and extension stop carrier disks 28, 29, a clamping rod 32 with a cross yoke 33, an eccentric element 34 (FIGS. 8A, 8B), and the manually actuated clamping lever 11 which is connected torsionally strong to the eccentric element 34. The locking means is located except for the clamping lever 11 in the front cavity 35 (FIG. 2) of the housing 12.

The clamping rings 30, 31 of the locking means have an inside diameter which corresponds essentially to the outside diameter of the flexion and extension stop carrier disks 28, 29, so that with little play they can be seated on the flexion and extension stop carrier disks 28, 29. In the seated state the clamp rings 30, 31 are thus in the same plane as the flexion and extension stop carrier disks 28, 29. The thickness of the clamping rings 30, 31 corresponds roughly to that of the flexion and extension stop carrier disks 28, 29. Furthermore the clamping rings 30, 31 each have a through slot 36 and 37 so that the ends of the clamping rings 30, 31 can be drawn together or moved apart from one another. If the clamping rings 30, 31 are drawn together, they sit securely on the peripheral surface of the flexion and extension stop carrier disks 28, 29 so that they can no longer turn relative to the clamping rings 30, 31. If the clamping rings 30, 31 are spread, the flexion and extension stop carrier disks 28, 29 can turn freely. In the intermediate position in which the clamping rings 30, 31 are neither drawn together nor actively spread, the clamping rings 30, 31 as a result of their inherent tension rest only slightly on the flexion and extension stop carrier disks 28, 29 so that the flexion and extension stop carrier disks 28, 29 can be turned as a small force is applied.

The clamping rings 30, 31 are drawn together via the clamping rod 32 which is shown in the isolated position in FIG. 9 and which connects the cross yoke 33 to the eccentric element 34 (FIG. 8A). The clamping rod 32 in the assembled state of the orthesis is between the clamping rings 30, 31 which have a certain distance to one another. The cross yoke 33 extends over both clamping rings 30, 31 from overhead and lies in the semicircular recesses 38, 39 which are formed in the upper end of the clamping rings 30, 31. On the lower end the clamping rod 32 has an annular sleeve 40. This sleeve 40 is used to hold an eccentric surface 41 of the eccentric element 34 which is located eccentrically to the axis 42 of rotation of the eccentric element 34. Otherwise the eccentric element 34 in the area of its end 43 which is located at the bottom in FIG. 8A is supported in a through hole 44 of the rear clamping ring 30. The bearing surface 45 of the eccentric element 34 which is at the top in FIG. 8A sits in a through hole 46 of the front clamping ring 37 which is flush with the through hole 44 of the rear clamping ring 30. Furthermore, the eccentric element 34 has a forward bearing surface 47 which sits in the through hole 48 of a front housing cover 49. The eccentric element 34 thus cannot move, but is supported to be able to turn around its own axis 42 of rotation in the housing 12 of the orthesis and at the same time holds the clamping rings 30, 31 in place so that they are locked relative to the housing 12 and thus also relative to the second rail 2.

If the eccentric element 34 is turned by the clamping lever 11 such that the eccentric surface 41 moves down, the clamping rod 32 is likewise pulled down and draws the upper end of the clamping rings 30, 31 likewise down by means of the cross yoke 33, i.e. in the direction of the lower end of the clamping rings 30, 31. In this way the flexion and extension stop carrier disks 28, 29 are locked torsionally strong. If the clamping lever 35 is swivelled down (FIG. 1), the eccentric surface 41 of the eccentric element 34 moves up, by which the tension stress on the clamping rod 32 is cancelled. The flexion and extension stop carrier disks 28, 29 can at this point be turned with little expenditure of force in the manner to be described below. If the clamping lever 35 continues to be turned (to the left in FIG. 1), the clamping rod 32 continues to be pushed up, the clamping rings 30, 31 being spread by the transverse pins 60 which are located on the clamping rod 32 in the area of the slots 36, 37. In this way the frictional engagement between the clamping rings 30, 31 and the flexion and extension stop carrier disks 28, 29 is completely cancelled, so that the flexion and extension stop carrier disks 28, 29 can be moved without any hindrance.

The flexion and extension stop 26, 27 is adjusted by means of a stop driver means 51 which is shown in isolation in FIG. 4 and in the assembled state in FIG. 5. The stop driver means 51 is connected torsionally strong to the first rail 1 and swivels along with it. The stop driver means 51 consists of two stop driver disks 52, 53 which each have a lengthwise slot 54, 55 which can be routed in an arc shape around the swivelling axis 10. The lengthwise slots 54, 55 extend in the circumferential direction over an angle of roughly 180 degrees. The front stop driver disk 53 has a center, tubular bearing extension 56 which can be inserted into a tubular bearing extension 57 of greater diameter of the rear stop driver disk 52, as shown in FIG. 5. In the installed state the two stop driver disks 52, 53 are connected torsionally strong to one another, their being located parallel to one another with a certain distance. The two lengthwise slots 54, 55 are aligned flush to one another.

The extension stop carrier disk 29 is supported on the rear bearing extension 57 in the immediate vicinity of the rear stop driver disk 52, the extension stop 27 extending into the lengthwise slot 54. The flexion stop carrier disk 28 is supported on the front bearing collar 58 of the forward stop driver disk 53 in its immediate vicinity, the flexion stop 26 projecting into the lengthwise slot 55. The flexion and extension stops 26, 27 have a length such that they extend completely through the adjacent lengthwise slot 55 and 54 and project even somewhat into the remote lengthwise slot 54 or 55. The flexion stop 26 and extension stop 27 thus bridge the intermediate space between the two stop driver disks 52, 53. As a result of this arrangement it is possible, in the intermediate space between the stop driver disks 52, 53, to provide a return spring 61 (FIG. 4) which engages both the flexion stop 26 and also the extension stop 27 and tries to force them back into the initial position, i.e. to turn the flexion stop carrier disk 28 and the extension stop carrier disk 29 back into the initial position in which the flexion stop 26 and the extension stop 27 are offset by 180 degrees to one another. This initial position is shown in FIGS. 6 and 7 by broken lines. In this initial position the flexion stop 26 rests on one end of the lengthwise slots 54, 55 and the extension stop 27 on the other end of the lengthwise slots 54, 55. In order to return the flexion stop 26 and the extension stop 27 into their initial positions shifted by 180 degrees, it is necessary for the clamping rings 30, 31 to be spread and thus lifted off the flexion stop carrier disk 28 and the extension stop carrier disk 29. As already stated, this spreading takes place by the clamping lever 11 being swivelled to the maximum degree down and to the left (as shown in FIG. 1), by which the eccentric element 34 lifts the clamping rod 32 and the transverse pins of the clamping rod 32 widen the slots 36, 37.

In order to position the flexion stop 26 and the extension stop 27 at the desired location, first the clamping lever 11 is swivelled into a neutral position, by which the clamping rod 32 applies neither tension nor compression on the clamping rings 30, 31. The clamping rings 30, 31, as a result of their inherent tension, now press a certain amount on the flexion and extension stop carrier disks 28, 29 so that they can still be turned, but no longer turn automatically back into their initial position based on the spring force of the return spring which is not shown. If at this point the first rail 1 together with the stop driver disks 52, 53 which are coupled torsionally strong to it is swivelled from the extension position, for example the one shown in FIG. 7, as far as the desired maximum flexion position as shown in FIG. 6, the flexion stop 26 is entrained by the stop driver disks 52, 53, as is shown by the arrow 59 in FIG. 6, since the flexion stop 26 strikes the end of the lengthwise slot 54, 55. The extension stop 27 remains with this swivelling motion in the flexion direction first of all in place, since it is located in the slot area without striking one of the two slot ends.

If at this point the first stop 1 is swivelled back from the maximum flexion position shown in FIG. 6 into the desired maximum extension position, as is shown in FIG. 7, the extension stop 27 is entrained by the stop driver disks 52, 53 in the direction of the arrow 60, since the extension stop 27 strikes the end of the lengthwise slots 54, 55. The flexion stop 26 remains in place with this swivelling motion in the extension direction since it is located in the free slot area, does not strike any end of the lengthwise slots 54, 55, and moreover is changed in turning back into the initial position by the small inherent clamping force of the clamping ring 31 which surrounds the flexion stop carrier disk 28.

In the position which is shown in FIG. 7, both the flexion stop 26 and also the extension stop 27 are at the desired points for limiting the swivelling area in the flexion and extension direction.

The position of the flexion stop 26 and the extension stop 27 is now locked by the clamping lever 11 being swivelled back into its clamped position. i.e. to the right in FIG. 1. In this way the clamping rod 32 is pulled down via the eccentric element 34 and the two clamping rings 30, 31 are drawn together. The flexion stop and extension stop carrier disks 28, 29 thus sit securely within the clamping rings 30, 31 and are connected torsionally strong to the housing and thus also to the second rail 2. After this locking of the flexion stop 26 and the extension stop 27, the first rail 1 can only be swivelled farther in the flexion direction until the end of the lengthwise slots 54, 55 strikes the flexion stop 26, as shown in FIG. 6. Furthermore, the first rail 1 can only be swivelled further in the extension direction until the end of the lengthwise slots 54, 55 strikes the extension stop 27, as shown in FIG. 7.

If the limits of the swivelling range are to be reset, it is simply necessary to release the clamping lever 11, whereupon the flexion stop and extension stop 26, 27 can be entrained again to the new limits of the swivelling range.

Using FIGS. 3A-3C, the action of the double acting spring force mechanism is detailed below. For the sake of clarity, in these schematics in the area of the rail hinge 3 only the worm wheel 14 with the coupling point 24 of the connecting rod 15 is shown.

In the position shown in FIG. 3 Autotransporter the orthesis is in the flexion range in which the first rail 1, due to the transverse force of the compression spring 18 which is transferred via the connecting rod 15 to the coupling point 24 which is located off-center on the worm wheel 14, is displaced in the flexion direction, i.e. counterclockwise.

If at this point the patient extends his forearm and swivels the first rail 1 clockwise, he must first overcome the increasing pretensioning force of the compression spring 18, since in this motion the compression spring 18 is compressed.

The compression spring 18 is compressed until the swivelling angle shown in FIG. 3B is reached. At this swivelling angle which is called dead center, the connecting rod 15 is in a straight line from the compression spring 18 to the swivelling axis 10. The transverse force of the compressions spring 18 thus travels through the swivelling axis 10 such that no torque is transferred to the first rail 1.

If the patient extends his forearm farther and thus the first rail 1 beyond dead center in the extension direction, as shown in FIG. 3C, a torque again begins immediately to act on the first rail 1, since now the transverse force of the compression spring 18 no longer moves through the swivelling axis 10, but laterally past it.

Depending on in which swivelling angle range with respect to dead center the first rail 1 is located relative to the second rail 2, the compression spring 18 thus applies torque in the flexion and extension direction.

With respect to the double acting spring force mechanism, reference is also made to German patent application no. 199 04 554.2, specifically to its disclosure contents.

Alternatively to the described embodiment, it is easily possible to make the locking means for the flexion and extension stop carrier disks 28, 29 not frictionally engaged as a clamping means, but by adhesion due to the configuration of the flexion and extension stop carrier disks 28, 29 as gears. Furthermore, it is also possible to provide the clamping surfaces between the flexion and extension stop carrier disks 28, 29 and the clamping rings 30, 31 with coatings which increase friction.

1. Orthesis for reducing extension and/or bending deficits of a first extremity relative to a second extremity which is hinged to the first extremity, with
a first rail (1) which can be attached to the first extremity,
a second rail (2) which can be attached to the second extremity,
a rail hinge (3) which hinges the first rail (1) and the second rail (2) to one another,
a spring force mechanism which acts between the first rail (1) and the second rail (2) in order to swivel the first rail (1) relative to the second rail (2),
a flexion stop (26) which can be locked relative to the second rail (2) for limiting the swivelling range in the flexion direction,
an extension stop (27) which can be locked relative to the second rail (2) for limiting the swivelling range in the extension direction, characterized in that
the flexion stop (26) is located on a flexion stop carrier disk (28) which can be turned around the swivelling axis (10) of the rail hinge (3),
the extension stop (27) is located on the extension stop carrier disk (29) which can be turned around the swivelling axis (10) of the rail hinge (3),
the flexion stop carrier disk (28) and extension stop carrier disk (29) are dynamically connected to a releasable locking means in order to block or enable the rotation of the flexion stop carrier disk (28) and extension stop carrier disk (29) around the swivelling axis (10),
there is a stop driver means (51) which is coupled torsionally strong to the first rail (1), which can turn together with it around the swivelling axis (10), and with which with the locking means released the flexion stop (26) can be moved by swivelling the first rail (1) as far as the desired limit of the flexion range and the extension stop (27) can be moved by swivelling the first rail (1) as far as the desired limit of the extension swivelling range, and
by locking the locking means the flexion stop (26) can be locked at the limit of the flexion swivelling range and the extension stop (27) can be locked at the limit of the extension swivelling range.

2. Orthesis as claimed in claim 1, wherein the stop driver means (51) which is coupled torsionally strong to the first rail (1) is also made as a swivelling range limitation means which at the limit of the flexion swivelling range strikes the flexion stop (26) and at the limit of the extension swivelling range strikes the extension stop (27).

3. Orthesis as claimed in claim 1 or 2, wherein the stop driver means (51) consists of at least one stop driver disk (52, 53) with a lengthwise slot (54, 55) which is routed in an arc shape around the swivelling axis (10) and into which the flexion stop (26) and extension stop (27) project.

4. Orthesis as claimed in one of the preceding claims, wherein there is a return spring (61) which engages the flexion stop carrier disk (28) and the extension stop carrier disk (29) in order to return the flexion stop (26) and extension stop (27) to their initial location when the locking means has been released.

5. Orthesis as claimed in one of the preceding claims, wherein the locking means consists of a clamping means which is held on the second rail (2), which extends over the flexion stop carrier disk (28) and the extension stop carrier disk (29), and which can be moved by a common clamp actuating means between the clamp position and the release position.

6. Orthesis as claimed in one of the preceding claims, wherein the clamp means feasibly consists of two clamping rings (30, 31) which extend over the flexion stop carrier disk (28) and the extension stop carrier disk (29) on their outer peripheral surface with narrow play and in the clamped position prevent rotation of the flexion stop carrier disk (28) and the extension stop carrier disk (29).
